SEQUENCE LISTING (23 sequences)

SEQ ID NO: 1 - protein, 10 aa, Homo sapiens
PGKNGERGGP

SEQ ID NO: 2 - protein, 11 aa, Homo sapiens
APGKNGERGGP

SEQ ID NO: 3 - protein, 9 aa, Homo sapiens
GKNGERGGP

SEQ ID NO: 4 - protein, 10 aa, Mus musculus
SASSSVSYLN

SEQ ID NO: 5 - protein, 7 aa, Mus musculus
STSNLAS

SEQ ID NO: 6 - protein, 9 aa, Mus musculus
HQRSSYPPT

SEQ ID NO: 7 - protein, 16 aa, Mus musculus
RSSQNIVHRDGNTYLE

SEQ ID NO: 8 - protein, 7 aa, Mus musculus
RVSNRFS

SEQ ID NO: 9 - protein, 9 aa, Mus musculus
FQGSHVPWT

SEQ ID NO: 10 - protein, 7 aa, Mus musculus
TSGMGVG

SEQ ID NO: 11 - protein, 16 aa, Mus musculus
HIYWDDDKYYNPSLKS

SEQ ID NO: 12 - protein, 9 aa, Mus musculus
NLLPGGFAY

SEQ ID NO: 13 - protein, 73 aa, Mus musculus
SASSSVSYLNWFQQKPGTSPKLWIYSTSNLASGVPARFSGSGSGTSYSLTISRMEAEDAATCNWHQRSSYPPT

SEQ ID NO: 14 - protein, 79 aa, Mus musculus
RSSQNIVHRDGNTYLEWYLQKPGQSPILLIDRVSNRFSGVPDRFSGSGSGTDFTLKISRVEAEDLGVYYCFQGSHVPWT

SEQ ID NO: 15 - protein, 78 aa, Mus musculus
TSGMGVGWIRQPSGRGLEWLAHIYWDDDKYYNPSLKSQLTISKDTSRNQVFLKITSVDTADTATYYCARNLLPGGFAY

SEQ ID NO: 16 - protein, 112 aa, Mus musculus
DVVMTQTPLSLPVSLGDQASISCRSSQSLVHSNGNTYLHWFLQKPGQSPKLLIYKVSNRFSGVPDRFSGSGSGTDFTLKISRVEAEDLGVYFCSQSTHVPYTFGGGTKLEIK

SEQ ID NO: 17 - protein, 112 aa, Mus musculus
DVLMTQTPLSLRVSLGDQASISCRSSQNIVHRDGNTYLEWYLQKPGQSPILLIDRVSNRFSGVPDRFSGSGSGTDFTLKISRVEAEDLGVYYCFQGSHVPWTFGGGTKLEIK

SEQ ID NO: 18 - protein, 117 aa, Mus musculus
QVTLKESGPGILKPSQTLSLTCSFSGFSLSTSGMGVGWIRQPSGRGLEWLAHIYWDDDKYYNPSLKSQLTISKDTSRNQVFLKITSVDTADTATYYCARNLLPGGFAYWGQGTLVTV

SEQ ID NO: 19 - protein, 13 aa, synthetic construct
  PEPTIDE 13: note = KLH - keyhole limpethemocyanin carrier protein
PGKNGERGGPGGC

SEQ ID NO: 20 - protein, 10 aa, synthetic construct
  PEPTIDE 10: note = optional linker to Biotin conjugate
PGKNGERGGP

SEQ ID NO: 21 - protein, 467 aa, Mus musculus
MDRLTSSFLLLIVPAYVLSQVTLKESGPGILKPSQTLSLTCSFSGFSLSTSGMGVGWIRQPSGRGLEWLAHIYWDDDKYYNPSLKSQLTISKDTSRNQVFLKITSVDTADTATYYCARNLLPGGFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 22 - protein, 235 aa, Mus musculus
MHFQVQIFSFLLISASVIMSRGQIVLTQSPAIMSTCPGEKVTITCSASSSVSYLNWFQQKPGTSPKLWIYSTSNLASGVPARFSGSGSGTSYSLTISRMEAEDAATCNWHQRSSYPPTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 23 - protein, 238 aa, Mus musculus
MKLPVRLLVLMFWIPGSSSDVLMTQTPLSLRVSLGDQASISCRSSQNIVHRDGNTYLEWYLQKPGQSPILLIDRVSNRFSGVPDRFSGSGSGTDFTLKISRVEAEDLGVYYCFQGSHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2026/0309721 A1
(43) Pub. Date: Oct. 8, 2026

(54) C3-HNE Assay

(71) Applicant: Nordic Bioscience A/S, Herlev (DK)

(72) Inventors: Thomai Tsapanou Katranara, Copenhagen SV (DK); Joachim Høg Mortensen, Hundige Strand (DK); Marta Alexdottir, Copenhagen S (DK); Morten A. Karsdal

(73) Assignee: Nordic Bioscience A/S, Herlev (DK)

(21) Appl. No.: 19/485,643

(22) PCT Filed: May 20, 2024
(86) PCT No.: PCT/EP2024/063840

§ 371 Date: Nov. 18, 2025

(30) Foreign Application Priority Data
May 19, 2023 (GB) 2307576.5

(51) Int. Cl. G01N 33/68; C07K 16/18

(52) U.S. Cl. CPC G01N 33/6893; C07K 16/18; C07K 2317/565; G01N 2470/04; G01N 2470/10; G01N 2800/065

(57) ABSTRACT

The present invention relates to methods of immunoassay for detecting HNE-generated fragments of the α1 chain of type III collagen in a patient sample, and the use thereof for detecting and/or monitoring inflammatory bowel disease (IBD) or a particular level of severity thereof in a patient. The present invention also relates to monoclonal antibodies and assay kits for use in said methods of immunoassay.

### FIELD OF THE INVENTION

The present invention relates to methods of immunoassay for detecting HNE-generated fragments of the α1 chain of type Ill collagen in a patient sample, and the use thereof for detecting and/or monitoring inflammatory bowel disease (IBD) or a particular level of severity thereof in a patient. The present invention also relates to monoclonal antibodies and assay kits for use in said methods of immunoassay.

### BACKGROUND

Inflammatory bowel disease IBD is an umbrella term for chronic inflammatory disorders in the gastrointestinal (GI) tract, characterized by damage in the epithelial barrier. The location and depth of the damage categorizes the disease into Crohn's disease (CD) or Ulcerative Colitis (UC) (Jostins et al., 2012, Graham et al., 2020). The chronic disease develops due to genetic susceptibility and activation of the immune response. When an abnormal immune response to the gut microbiota occurs, excessive intestinal damage and mucosal inflammation follows (Qiu et al., 2022). Simultaneously, uncontrolled and high proteolytic activity has been linked to IBD, where proteases are secreted from a variety of immune cells (such as neutrophils, lymphocytes and macrophages) (Curciarello et al., 2020, Kriaa et al., 2021).

The intestinal wall is an integral component of protection for the GI tract, maintaining homeostasis through interactions between epithelial and stromal cells (Kong et al., 2018). Epithelial and stromal cells jointly produce and organize the intestinal extracellular matrix (ECM). Type III collagen is expressed throughout all layers of the intestinal mucosa, and it is the second most abundant collagen (20%) in the intestine, synthesized mainly by fibroblasts (Graham 1988). ECM remodeling is a dynamic process that requires interactions between the resident cells and the ECM components, resulting in the secretion of proteolytic enzymes. Healthy tissues are continuously and steadily being remodeled; however, an abnormal regulation can cause an imbalance in the ECM turnover, leading to chronic inflammation (Kehlet et al., 2018). Inflammation is the biological response to harmful stimuli and the inflammatory response can be acute or chronic (Signore et al., 2013, Pahwa et al., 2021). Acute inflammation is mediated by granulocytes such as neutrophils, which play an essential role in chronic inflammation. Chronic inflammation of the gut mucosa involves granule release and activation of neutrophils (NETosis) that form neutrophil extracellular traps (NETs). NETs are comprised of DNA threads laced with nuclear proteins, such as histones, and cytotoxic granule components such as human neutrophil elastase (HNE) (Yuen et al., 2016, Menegazzi et al., 2012). HNE is a serine protease that cleaves ECM components, such as elastin and collagens, and upregulates several pro-inflammatory cytokines (Curciarello et al., 2020). Therefore, excessive recruitment of neutrophils can lead to tissue damage in the inflamed mucosa (Saez et al., 2023). Neutrophil presence and activity are increased in IBD patients, and HNE levels are significantly higher in the plasma of IBD patients than in healthy individuals (Hansberry et al., 2017, Fischbach et al., 1987). Increased levels of HNE are detected in both UC and CD, and the ratio in expression between HNE and antiproteases (such as elafin) is significantly shifted towards the HNE in CD compared with UC (Schmid et al., 2007). Furthermore, HNE induces proteolytic degradation of therapeutic antibodies in IBD patients, leading to non-responsiveness to treatment (Curciarello et al., 2020).

### SUMMARY OF THE INVENTION

A collagen type III fragment derived by the neutrophil-specific protease HNE has been identified which reflects neutrophil-derived mucosal damage and can be used as a direct measure of immune cell activity. A competitive enzyme-linked immunosorbent assay (ELISA) targeting an HNE-generated neo-epitope of the α1 chain of type III collagen has been developed, and has demonstrated the utility of the HNE-generated neo-epitope as a biomarker for inflammatory bowel disease (IBD) in general, and early-stage IBD in particular.

Accordingly, in a first aspect the present invention provides a method of immunoassay, said method comprising contacting a patient sample with a monoclonal antibody that specifically binds to an HNE-generated neo-epitope of an HNE-generated fragment of the α1 chain of type III collagen, and detecting and determining the amount of binding between the monoclonal antibody and peptides in the sample, wherein the HNE-generated neo-epitope consists of an N-terminus sequence of the HNE-generated fragment at an end of the HNE-generated fragment that has been cleaved by HNE.

In a preferred embodiment, the method of immunoassay comprises;
i) contacting a patient sample with a monoclonal antibody that specifically binds to the N-terminus amino acid sequence PGKNGERGGP (SEQ. ID NO: 1) (also referred to herein as “C3-HNE”, or the “C3-HNE target sequence”); and
ii) detecting and determining the amount of binding between said monoclonal antibody and peptides in the sample.

In a preferred embodiment, the method of immunoassay is a method of immunoassay for detecting and/or monitoring inflammatory bowel disease or a particular level of severity thereof in a patient, the method further comprising correlating said amount of binding with values associated with normal healthy subjects and/or values associated with known disease severity and/or values obtained from said patient at a previous time point and/or with a predetermined cut-off value.

In a preferred embodiment, the inflammatory bowel disease (IBD) is Crohn's disease (CD) or ulcerative colitis (UC).

In a preferred embodiment, the method is a method of immunoassay for detecting early stage inflammatory bowel disease.

In a preferred embodiment, the patient sample is a human biofluid sample. Preferably the sample is a blood-based sample, such as blood (whole blood), plasma or serum.

Where the monoclonal antibody is a monoclonal antibody that specifically binds to the N-terminus amino acid sequence PGKNGERGGP (SEQ. ID NO: 1), the monoclonal antibody preferably does not specifically bind to a peptide having the N-terminus amino acid sequence APGKNGERGGP (SEQ. ID NO: 2) (i.e. an elongated version of the C3-HNE target sequence extended at its N-terminus by the addition of an alanine residue) and/or does not specifically bind to a peptide having the N-terminus amino acid sequence GKNGERGGP (SEQ. ID NO: 3) (i.e. a truncated version of the C3-HNE target sequence truncated by removal of the first proline residue). Preferably, the ratio of the affinity of said antibody for the C3-HNE target sequence to the affinity of said antibody for the elongated version of the target sequence is at least 10 to 1, and more preferably is at least 20 to 1 or at least 30 to 1.

The term “specifically bind” as used herein means that the antibody binding is selective for the antigen and that this binding can be distinguished from unwanted or non-specific interactions. The ability of a monoclonal antibody to bind to a specific epitope or peptide sequence can be measured either through an enzyme-linked immunosorbent assay (ELISA) as described herein or other techniques familiar to one of skill in the art, e.g. surface plasmon resonance (SPR) technique (analyzed e.g. on a BIAcore instrument) and traditional binding assays. The extent of binding of a monoclonal antibody to an unrelated protein is less than about 10% of the binding of the monoclonal antibody to the epitope or peptide as measured, e.g., by ELISA. “Affinity” refers to the strength of the sum total of non-covalent interactions between a single binding site of a molecule (e.g., a epitope binding region of an antibody) and its binding partner (e.g., an epitope or antigen). Unless indicated otherwise, as used herein, “binding affinity” refers to intrinsic binding affinity which reflects a 1:1 interaction between members of a binding pair (e.g., an antigen binding moiety and an antigen). The affinity of a molecule for its partner can generally be represented by the dissociation constant (Kd), which is the ratio of dissociation and association rate constants (koff and kon, respectively). Thus, equivalent affinities may comprise different rate constants, as long as the ratio of the rate constants remains the same. The dissociation constant represents the concentration of the antigen at which half of the binding sites on the antibody are occupied. A lower Kd indicates a higher binding affinity between the antibody and antigen, while a higher Kd reflects weaker binding. Several methods are available to measure the Kd of an antibody, including surface plasmon resonance (SPR), isothermal titration calorimetry (ITC), and fluorescence-based assays. In certain aspects, a monoclonal antibody that binds to the epitope or peptide has a dissociation constant (KD) of <1 μM, <100 nM, <10 nM, <1 nM, <0.1 nM, <0.01 nM, or <0.001 nM (e.g. 10⁸ M or less, e.g. from 10⁸ M to 10¹³ M, e.g., from 10⁹ M to 10¹³ M).

Where the monoclonal antibody is a monoclonal antibody that specifically binds to the N-terminus amino acid sequence PGKNGERGGP(SEQ ID NO: 1), the monoclonal antibody may for example be raised against a synthetic peptide having the N-terminus amino acid sequence PGKNGERGGP(SEQ ID NO: 1). For example, the monoclonal antibodies may be raised by: (a) immunizing a rodent (or other suitable mammal) with a synthetic peptide comprising the N-terminus sequence PGKNGERGGP(SEQ ID NO: 1), which peptide may optionally be linked at its N-terminus to an immunogenic carrier protein (such as keyhole limpet hemocyanin); (b) isolating and cloning a single antibody producing cell; and (c) assaying the resulting monoclonal antibodies to ensure that they have the desired specificity. An exemplary protocol of the development, production and characterization of suitable monoclonal antibodies is described in the Examples section, infra.

In certain exemplary embodiments, where the monoclonal antibody is a monoclonal antibody that specifically binds to the N terminus amino acid sequence PGKNGERGGP(SEQ ID NO: 1), the monoclonal antibody may preferably comprise one or more complementarity-determining regions (CDRs) selected from:

CDR-La1:
(SEQ ID NO: 4)
SASSSVSYLN
CDR-La2:
(SEQ ID NO: 5)
STSNLAS
CDR-La3:
(SEQ ID NO: 6)
HQRSSYPPT
CDR-Lb1:
(SEQ ID NO: 7)
RSSQNIVHRDGNTYLE
CDR-Lb2:
(SEQ ID NO: 8)
RVSNRFS
CDR-Lb3:
(SEQ ID NO: 9)
FQGSHVPWT
CDR-H1:
(SEQ ID NO: 10)
TSGMGVG
CDR-H2:
(SEQ ID NO: 11)
HIYWDDDKYYNPSLKS
CDR-H3:
(SEQ ID NO: 12)
NLLPGGFAY

Preferably the monoclonal antibody comprises at least 2,3,4,5 or 6 of the above listed CDR sequences.

Preferably the monoclonal antibody has a light chain variable region comprising the CDR sequences:

CDR-La1:
(SEQ ID NO: 4)
SASSSVSYLN
CDR-La2:
(SEQ ID NO: 5)
STSNLAS
CDR-La3:
(SEQ ID NO: 6)
HQRSSYPPT

Alternatively, preferably the monoclonal antibody has a light chain variable region comprising the CDR sequences:

CDR-Lb1:
(SEQ ID NO: 7)
RSSQNIVHRDGNTYLE
CDR-Lb2:
(SEQ ID NO: 8)
RVSNRFS
CDR-Lb3:
(SEQ ID NO: 9)
FQGSHVPWT

Preferably the monoclonal antibody has a light chain that comprises framework sequences between the CDRs, wherein said framework sequences are substantially identical or substantially similar to the framework sequences between the CDRs in either of the light chain sequences below (in which the CDRs are shown in bold and underlined, and the framework sequences are shown in italics)

(SEQ ID NO: 13)
SASSSVSYLNWFQQKPGTSPKLWIYSTSNLASGVPARFSGSGSGTSYSL
TISRMEAEDAATCNWHQRSSYPPT
or
(SEQ ID NO: 14)
RSSQNIVHRDGNTYLEWYLQKPGQSPILLIDRVSNRFSGVPDRFSGSGS
GTDFTLKISRVEAEDLGVYYCFQGSHVPWT

Preferably the monoclonal antibody has a heavy chain variable region comprising the CDR sequences:

CDR-H1:
(SEQ ID NO: 10)
TSGMGVG
CDR-H2:
(SEQ ID NO: 11)
HIYWDDDKYYNPSLKS
CDR-H3:
(SEQ ID NO: 12)
NLLPGGFAY

Preferably the monoclonal antibody has a heavy chain that comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID NO: 15)
TSGMGVGWIRQPSGRGLEWLAHIYWDDDKYYNPSLKSQLTISKDTSRNQ
VFLKITSVDTADTATYYCARNLLPGGFAY

Preferably, the monoclonal antibody comprises the light chain variable region sequence:

(SEQ ID NO: 16)
DVVMTQTPLSLPVSLGDQASISCRSSQSLVHSNGNTYLHWFLQKPGQSP
KLLIYKVSNRFSGVPDRFSGSGSGTDFTLKISRVEAEDLGVYFCSQSTH
VPYT FGGGTKLEIK
or
(SEQ ID NO: 17)
DVLMTQTPLSLRVSLGDQASISCRSSQNIVHRDGNTYLEWYLQKPGQSP
ILLIDRVSNRFSGVPDRFSGSGSGTDFTLKISRVEAEDLGVYYCFQGSH
VPWTFGGGTKLEIK
(CDRs bold and underlined; Framework sequences in italics)

and/or the heavy chain variable region sequence:

(SEQ ID NO: 18)
QVTLKESGPGILKPSQTLSLTCSFSGFSLSTSGMGVGWIRQPSGRGLEW
LAHIYWDDDKYYNPSLKSQLTISKDTSRNQVFLKITSVDTADTATYYCA
RNLLPGGFAYWGQGTLVTVSA
(CDRs bold and underlined; Framework sequences in italics)

As used herein, the framework amino acid sequences between the CDRs of an antibody are “substantially identical” or “substantially similar” to the framework amino acid sequences between the CDRs of another antibody if they have at least 70%, 80%, 90% or at least 95% similarity or identity. The similar or identical amino acids may be contiguous or non-contiguous. The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, Ile, Val, Cys; Group 5 Phe Thy Trp.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

In preferred embodiments the immunoassay is a competition assay or a sandwich assay. The immunoassay may, for example, be a radio-immunoassay or an enzyme-linked immunosorbent assay (ELISA). Such assays are techniques known to the person skilled in the art. Most preferably the immunoassay is a competitive ELISA.

As used herein the term “N-terminus” refers to an N-terminal peptide sequence at the extremity of a polypeptide, i.e. at the N-terminal end of the polypeptide, and is not to be construed as meaning in the general direction thereof. As used herein the term “C-terminus” refers to a C-terminal peptide sequence at the extremity of a polypeptide, i.e. at the C-terminal end of the polypeptide, and is not to be construed as meaning in the general direction thereof.

As used herein, the terms “peptide” and “polypeptide” are used synonymously.

As used herein the term “monoclonal antibody” refers to both whole antibodies and to fragments thereof that retain the binding specificity of the whole antibody, such as for example a Fab fragment, F(ab′) 2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the present invention, a monoclonal antibody comprising any constant region known in the art can be used. In the case of mouse antibodies and human antibodies, the constant light chains are classified as either kappa or lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgGI, IgG2, IgG3, and IgG4 in the case of humans and IgGI, IgG2a, IgG2b, IgG2c and IgG3 in the case of mice. The monoclonal antibody may preferably be of the IgG isotype, including any one of the IgG subclasses.

The CDR of an antibody can be determined using methods known in the art such as that described by Kabat et al. Antibodies can be generated from B cell clones. The isotype of the antibody can be determined by ELISA specific for IgM, IgG or IgA isotype, or subclass. The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example, primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

As used herein the term “amount of binding” refers to the quantification of binding between the antibody and peptides in the patient sample. Said quantification may for example be determined by comparing the measured values of binding in the patient sample against a calibration curve produced using measured values of binding in standard samples containing known concentrations of a peptide to which the antibody specifically binds, in order to determine the quantity of peptide to which the antibody specifically binds in the patient sample. Any suitable analytical method can be used for measuring the amount of binding. For example, an ELISA method can be used in which spectrophotometric analysis is used to measure the amount of binding both in the patient samples and when producing the calibration curve.

As used herein the term “predetermined cut-off value” means an amount of binding that is determined statistically to be indicative of a high likelihood of a disease (e.g. IBD) or a particular severity thereof in a patient, in that a measured value of the target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the presence of said disease or particular severity thereof.

As used herein, the term “values associated with normal healthy subjects” means standardised quantities of binding determined by the method described supra for samples from subjects considered to be healthy, i.e. without disease (i.e. without IBD); and the term “values associated with known disease severity” means standardised quantities of binding determined by the method described supra for samples from patients known to have disease (i.e. an IBD) of a known severity.

In a second aspect, the present invention provides a method of treating inflammatory bowel disease (IBD) in a patient in need thereof, the method comprising:
(a) carrying out a method of immunoassay for detecting IBD or a particular level of severity thereof in accordance with the first aspect of the present invention on a sample from a patient; and
(b) administering to the patient a therapy for the treatment of IBD if it is determined in step (a) that the patient has IBD or said particular level of severity thereof.

Preferred embodiments of the method in accordance with the second aspect will be apparent from the foregoing discussion of preferred embodiments of the methods according to the first aspect. For example, the IBD may in particular comprise Crohn's disease (CD) or ulcerative colitis (UC); and/or step (a) may in particular comprise carrying out the method of immunoassay in accordance with the first aspect of the invention for detecting mild or early stage IBD and step (b) may then comprise administering to the patient a therapy for the treatment of IBD if it is determined in step (a) that the patient has mild or early stage IBD. Step (a) may comprise carrying out the method of immunoassay in accordance with the first aspect of the invention for detecting patients who are more likely to respond to treatment and step (b) may then comprise administering to the patient a therapy for the treatment of IBD if it is determined in step (a) that the patient is likely to respond to treatment. The patients may already have been diagnosed with an IBD, or mild or early stage IBD may also be detected by the immunoassay.

The therapy may be any therapy suitable for treating the inflammatory bowel disease in question. The therapy may for example comprise or consist of one or more medicaments, one or more lifestyle changes, one or more surgeries or combinations thereof. Medicaments may be formulated for topical or systemic administration. Topical medicaments may for example be formulated as creams, foams, gels, lotions, or ointments for administration. Systemic medicaments may for example be formulated for enteral or parenteral administration. Surgeries may be curative surgeries, preventative surgeries, palliative surgeries and/or restorative surgeries.

For example, where the inflammatory bowel disease is Crohn's disease suitable therapies may comprise one or more of: lifestyles changes such as dietary adjustments, elemental diet, proper hydration, smoking cessation; medicaments such as antibiotics, aminosalicylate anti-inflammatories, corticosteroids, 5-aminosalicylic acid (5-ASA), prednisone, azathioprine, 6-mercaptopurine, methotrexate, anti-TNF therapies (also referred to in the art as TNF inhibitors) such as monoclonal antibodies that inhibit the effects of TNF such as infliximab, adalimumab and certolizumab, and other biologics and antibodies such as vedolizumab, ustekinumab, and natalizumab; and combinations thereof.

Where the inflammatory bowel disease is ulcerative colitis suitable therapies may comprise one or more of: medicaments such as aminosalicylates such as Mesalazine, Sulfasalazine, Balsalazide, Olsalazine, corticosteroids such as Cortisone, Prednisone, Hydrocortisone, Methylprednisolone, Budesonide, immunosuppressive drugs such as Mercaptopurine, Azathioprine, Methotrexate; and anti-TNF therapies (TNF inhibitors) such as Infliximab, Adalimumab and Golimumab, and other biologics and antibodies such as Tofacitinib and Vedolizumab; surgical procedures such as a partial or total colectomy; and combinations thereof.

In a third aspect, the present invention provides a monoclonal antibody that specifically binds to the N-terminus amino acid sequence PGKNGERGGP (SEQ ID NO: 1) (i.e. the C3-HNE target sequence).

The antibody according to the third aspect of the invention is, in particular, suitable for use in carrying out the methods of immunoassay according to the first aspect of the invention. Preferred embodiments and features of the antibody according to the third aspect will therefore be apparent from the above discussion of the preferred embodiments of the methods according to the first aspect.

In a fourth aspect, the present invention provides an immunoassay kit comprising a monoclonal antibody in accordance with the third aspect of the present invention, and at least one of:
a streptavidin coated well plate
a biotinylated peptide PGKNGERGGP-L-Biotin (SEQ ID NO: 1) wherein L is an optional linker
a secondary antibody for use in a sandwich immunoassay
a calibrator protein comprising the N-terminus amino acid sequence PGKNGERGGP (SEQ ID NO: 1)
an antibody biotinylation kit
an antibody HRP labelling kit
an antibody radiolabelling kit

The immunoassay kit according to the fourth aspect of the invention is, in particular, suitable for use in carrying out the method of immunoassay according to the first aspect of the invention. Further preferred embodiments and features of the immunoassay kit according to the fourth aspect will therefore be apparent from the above discussion of the preferred embodiments of the methods according to the first aspect.

### FIGURES

FIG. 1: Pilot study: Acute DSS colitis rat SD model. The rats were divided in 6 groups and they were treated with DSS in A, B and C groups and regular water in D, E and F groups.

FIG. 2: Dose-dependent reactivity was seen for the mAb when incubated with the selection (standard) peptide, whereas no reactivity was exhibited for the mAb when incubated with the elongated and truncated peptides.

FIG. 3: C3-HNE is elevated in both samples with CD (p=0.0023) and UC (p=0.0361) compared to healthy samples. The data is presented as median. Kruskal-Wallis rank test with Dunn's correction for multiple comparisons was used to compare differences among the groups. Results are shown in a box plot (showing all points), median with interquartile range. Significance is marked with p<0.05=* and p<0.01=**.

FIG. 4: Serum levels of C3-HNE in cohort I. C3-HNE formation in CD, UC samples and healthy donors in cohort I. C3-HNE is elevated in both samples with CD (p=0.0072) and UC (p=0.0437) compared to healthy samples. Mann-Whitney U test was used to compare differences between two groups. Results are shown in a box plot (showing all points), median with an interquartile range. Significant difference is marked with p<0.05=*, p<0.01=**.

FIG. 5: Serum levels of C3-HNE in cohort III. C3-HNE is elevated in samples with IBD (p=0.0034) compared to healthy donors. All quantified levels of C3-HNE were above LLOQ. Mann-Whitney U test was used to compare differences among the groups. Results are shown in a box plot (showing all points), median with interquartile range. Significance is marked with p<0.01=**.

FIG. 6: C3-HNE is elevated before clinically apparent mucosal damage in the DSS-colitis rat model. A. Serum levels of C3-HNE (% relative to baseline) in rats that were treated with 5% DSS and regular water. Two-way ANOVA with multiple comparisons was used to compare differences among the groups. Test Significance is marked with p<0.05=*. B. The DAI score was monitored daily in rats that were treated with 5% DSS and regular water. The induced DSS-colitis model is marked with red, whereas the control group is marked with blue.

FIG. 7: Serum levels of C3-HNE upon type III collagen cleavages. C3-HNE formation upon pre-cleavage of fibroblast-deposited type III collagen with MMP8 and MMP13 and final cleavage with HNE in a “Scar in a jar” (SIAJ) model.

FIG. 8: C3-HNE formation in conditioned media of primary neutrophils that were seeded in coated with type III collagen wells. Neutrophils were either inactivated or activated (LPS 25 ng/ml or 100 ng/ml) for 6 hours. IMDM was used as a reference control for inactivated cells and IMDM LPS 25 ng/ml & IMDM LPS 100 ng/ml were used as reference control for the activated, respectively.

FIG. 9: Serum levels of C3-HNE in patients with IBD in Cohort II and III, stratified according to response to treatment.

### EXAMPLES

The presently disclosed embodiments are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

### Monoclonal Antibody Production

Female Balb/C mice of 6-7 weeks of age were immunized by subcutaneous injections of 200 μl emulsified antigen and 100 μg of the C3-HNE [PGKNGERGGP-GGC-KLH (SEQ ID NO: 19)] neo-epitope peptide. The immunogen was prepared using 1:1 ratio of Sigma adjuvant system (Sigma©, Cat No S6322) and the KHL-conjugated immunogen. For 11 weeks, immunizations were performed at multiple sites on the abdomen area, consecutively at 2-week intervals. At this stage B-cells differentiate into plasma B-cells and memory B-cells. The mouse with the highest serum titer, thus the highest production of antibodies, was chosen for fusion and subsequently rested for one month. The mouse was then boosted intravenously with 50 μg/100 μl of the respective immunogenic peptide in saline solution. Spleen cells and SP2/0 myeloma cells were fused in polyethyleneglycol (PEG) to generate immortalized hybridoma cells that produce monoclonal antibodies, as described by Gefter et al., 1977. The fused cells were selected upon growth in a hypoxanthine-aminopterin-thymidine (HAT) medium. A collection of supernatants was performed for reactivity to the neo-epitope using an indirect ELISA with streptavidin-coated microtiter plates and biotinylated peptide for C3-HNE [PGKNGERGGP-LYS-Biotin] (SEQ ID NO: 20). The reactivity of the clones towards the elongated peptide [APGKNGERGGP (SEQ ID NO: 2)] and truncated peptide [_GKNGERGGP (SEQ ID NO: 3)] was tested, and the optimal clones were selected for subsequent amplification.

Antibody isotype was determined using isotype-specific anti-sense primers or universal primers following the technical manual of SMARTScribe™ Reverse Transcriptase Kit. (Takara, Cat. No.: 639537). All procedures were performed according to the manufacturers' instructions.

The isotype, sequence and CDRs of this monoclonal antibody were determined. Two alternative sequences for the light chain were detected. Even though a monoclonal antibody might be expected to produce a single light chain sequence, it is common for about 30% of monoclonal antibody clones to produce additional sequences (Bradbury et al (2018)). The sequence of the chains are as follows (CDRs underlined and in bold; N-terminus signal peptide and C-terminus Constant region in italics):

Heavy Chain Sequence (Mouse IgG2a isotype)
(SEQ ID NO: 21)
MDRLTSSFLLLIVPAYVLSQVTLKESGPGILKPSQTLSLTCSFSGFSLS
TSGMGVGWIRQPSGRGLEWLAHIYWDDDKYYNPSLKSQLTISKDTSRNQ
VFLKITSVDTADTATYYCARNLLPGGFAYWGQGTLVTVSAAKTTAPSVY
PLAPVCGDTTGSSVTLGCLVKGYFPEPVTLTWNSGSLSSGVHTFPAVLQ
SDLYTLSSSVTVTSSTWPSQSITCNVAHPASSTKVDKKIEPRGPTIKPC
PPCKCPAPNLLGGPSVFIFPPKIKDVLMISLSPIVTCVVVDVSEDDPDV
QISWFVNNVEVHTAQTQTHREDYNSTLRVVSALPIQHQDWMSGKEFKCK
VNNKDLPAPIERTISKPKGSVRAPQVYVLPPPEEEMTKKQVTLTCMVTD
FMPEDIYVEWTNNGKTELNYKNTEPVLDSDGSYFMYSKLRVEKKNWVER
NSYSCSVVHEGLHNHHTTKSFSRTPGK
Light Chain Sequence (Mouse Kappa Isotype)
(SEQ ID NO: 22)
MHFQVQIFSFLLISASVIMSRGQIVLTQSPAIMSTCPGEKVTITCSASS
SVSYLNWFQQKPGTSPKLWIYSTSNLASGVPARFSGSGSGTSYSLTISR
MEAEDAATCNWHQRSSYPPTFGAGTKLELKRADAAPTVSIFPPSSEQLT
SGGASVVCFLNNFYPKDINVKWKIDGSERQNGVLNSWTDQDSKDSTYSM
SSTLTLTKDEYERHNSYTCEATHKTSTSPIVKSFNRNEC
Or
(SEQ ID NO: 23)
MKLPVRLLVLMFWIPGSSSDVLMTQTPLSLRVSLGDQASISCRSSQNIV
HRDGNTYLEWYLQKPGQSPILLIDRVSNRFSGVPDRFSGSGSGTDFTLK
ISRVEAEDLGVYYCFQGSHVPWTFGGGTKLEIKRADAAPTVSIFPPSSE
QLTSGGASVVCFLNNFYPKDINVKWKIDGSERQNGVLNSWTDQDSKDST
YSMSSTLTLTKDEYERHNSYTCEATHKTSTSPIVKSFNRNEC

### Antibody Purification

The clone supernatant was purified using affinity chromatography with 1 ml HiTrap Protein G column (GE Healthcare Life Science, Little Chalfront, Buckinghamshire, UK). Protein G is a recombinant protein from Escherichia coli that binds to Immunoglobulin G (IgG) in eukaryotic species. Before starting the purification, the Protein G column was connected to the peristaltic pump, and washed with 20% ethanol, followed by Mili Q water washes. A second column wash followed with a 1 ml/min flow rate and 10 column volumes of washing buffer (0.02 M Natrium phosphate pH 7) to remove storage preservations. The procedure was split into two days, running 250 ml of supernatant through the column each time. Thus, 250 ml of sterile-filtered hybridoma supernatant was applied to the column, followed by a washing round of ten column volumes of washing buffer to eliminate unspecific binding proteins. The antibody was collected by adding elution buffer (0.1 Glycine, pH 2.7) to the column. The low pH of the elution buffer disrupts the binding between the mAb and protein G. The eluted mAbs were collected in four Eppendorf tubes containing 100 μl neutralization buffer (1 M Tris-HCl, pH 9.0), providing stability to the antibody. The concentration of the eluted mAb (IgG measurement) was measured on NanoDrop (NanoDrop 1000, Thermo Scientific). After measurement, all vials that contained more than 1 mg/ml of antibody were pooled together. The mAb was desalted using a 5 ml spin column (Zeba spin desalting column 7k MWCO, Thermo Scientific) was used. To exchange the storage solution, the column was centrifuged and submerged three times with Dulbecco's phosphate buffered saline (DPBS, Thermo Scientific). Then, the antibody was run through the column and collected in a clean tube. On the second day, this process was repeated. The purified and desalted mAb from both days was pooled, the absorbance was measured on NanoDrop, aliquoted in 500 μl per Eppendorf tube, and stored at −80° C.

### C3-HNE Assay Conditions

The C3 ELISA assay protocol was established as follows: A white streptavidin-coated 96 well plate (biomat, LOT: SA2758) was coated with 100 μl of 0.5 ng/ml synthetic biotinylated peptide for C3-HNE [PGKNGERGGP-LYS-Biotin (SEQ ID NO: 20)] in assay buffer (50 mM PBS-BTB, 8 g NaCl, pH 7.4) and incubated for 30 mins at 20° C. on shaker at 300 rpm. After washes with the washing buffer (25 mM Tris, 50 mM NaCl, 0.1% (v/v) Tween-20, pH 7.2), 1000 ng/ml standard (selection) peptide (PGKNGERGGP(SEQ ID NO: 1)) was serially diluted 11 times (two-fold) and added to the plate with 20 μl/well. Consequently, 20 μl of samples, controls (CO1 and CO2) and quality controls (QC1 and QC2, serum/supernatant samples obtained from BioVit (Westerbury, NY, USA) or Nordic Bioscience)) were added in double determination. Simultaneously, 100 μl of 10 ng/ml horse-radish peroxidase-conjugated monoclonal antibody were added and the plate was incubated for 20 hours at 4° C. in the dark on shaker at 300 rpm. The ECL Substrate was prepared 15-25 mins before use, according to the manufacturer's instructions (BM Chemiluminescence ELISA Substrate POD, Roche). A washing step was repeated, and 100 μl/well of the equilibrated ECL Substrate was added and incubated for 3 mins in the dark. The plate was read in ELISA reader (SpectraMax M5; Molecular Devices, UK) using Softmax Software under LUM mode (all wavelengths). Standard curves were plotted using a four-parametric mathematical fit model.

### Technical Validation

A variety of technical validation experiments were performed to assess the robustness, precision, interference, and stability of the assay. The lower limit of buffer (LLOB) was determined by performing a single run of 60 replicates of assay buffer. LLOB is defined as 20+3×SD of assay buffer with no acceptance criteria. The lower limit of quantification (LLOQ) is defined as the lowest analyte concentration in serum where the CV % of the precision profile equals 25%. The lower limit of quantification (LLOQ) was determined by five repetitions of runs with four low concentrated samples, two below and two above the estimated LLOQ.

### Assay Linearity

Assessing linearity, determines the precision of the assay and simultaneously evaluates the minimum required dilution (MRD) of the sample. It is essential that the MRD is established at the beginning of the assay validation to check whether there are matrix effects in undiluted samples or if the recovery of the diluted samples is not accepted. Thus, dilution of minimum three high concentrated serum samples (above IC50) was assessed for assay linearity. The linearity is accepted if the sample dilution curve follows the same pattern as the standard curve. The acceptance criteria for the dilution recovery was 100±20%, a measurement equal to the multiple of the resulting concentration with the dilution factor

$$( \frac{Concentration of Diluted Sample * Dilution Factor}{Concentration of Undiluted Sample} * 100 ) .$$

The samples were diluted in the assay buffer 2-fold, 4-fold and 8-fold and the protocol was repeated three times.

### Assay Accuracy

The accuracy of the assay is determined by the matrix-matrix spiking recovery. The test assesses whether the measured concentration of the samples is close to the real concentration of the complex matrix. This test uses three sample sets of serum samples with high (above IC50) and low (around IC20) analyte concentrations, spiked in different ratios. Each sample set had a matrix-matrix spike-in of the ratios 100:0, 75:25, 50:50, 25:75, and 0:100 of high to low concentration samples, respectively. Recovery percentage was calculated based on expected and measured concentrations. The accepted criteria for the recovery range were 100±20%.

### Assay Precision and Reproducibility

The precision of the assay is defined by the variance between sample replicates, either within the same plate or between plates. The reproducibility of the assay needs to be established ensuring that the quantified data is comparable and does not vary. The intermediate precision (inter-assay) provides information on the robustness of the assay's standard curve and how precise the measured concentrations of the sample replicates are between runs, while the intra-assay variation measures the variability within the plate. Ten independent runs were conducted with 8 samples ranging from IC20 to IC80, covering the assay's measurement range. The upper limit of quantification (ULOQ) was calculated as the mean of the highest standard curve point across the runs. The robustness of the assay was calculated as the coefficient of variation in percentage (CV %) of the sample measurements. The accepted CV % for the inter-assay and intra-assay variation is <15% and <10%, respectively. If the CV % for both parameters is low, the assay is considered robust.

### Assay Interference

Interference testing is a procedure required to evaluate whether different factors can affect the quantification of the samples, to avoid a false alteration of the analytic test result. The parameters that can affect the assay's precision is how the serum samples were collected and stored, the temperature of the kit reagents and the existence of endogenous or exogenous substances in the matrix. Thus, a complete standardization of the interference protocols is not feasible. The most frequent endogenous analytes that cause interference in ELISAs, are haemoglobin, biotin and lipids, and these establish the minimum required interference testing. Three haemoglobin samples of high (5 ng/ml), middle (2.5 ng/ml) and low (0 ng/ml) concentration, three lipid samples of high (5 ng/ml), middle (1.5 ng/ml) and low (0 ng/ml) concentration and eight biotin samples with a concentration ranging from 0 ng/ml to 100 ng/ml were spiked into three different samples (IC30-IC70). The spike-in interference was diluted 1:20 for haemoglobin, biotin, and lipid by adding 5 μL of each interference substance to 95 μL of serum. The accepted recovery criteria were 100±20%.

### Assay Stability

The purpose of the stability tests is to assess the stability of both the endogenous analytes, conducting a sample freeze-thaw test and an assay stability test. The freeze-thaw procedure determines the stability of the analyte in the intended matrix through five cycles of freezing and thawing. If all samples after the cycles are measured to be similar, there is no substantial variability. Thus, three samples (IC25−IC50) underwent freeze-thaw cycles at −20° C. in 12-hour intervals. The thawing step lasted for at least 30 mins. Upon completion of the freeze-thaw cycles, all aliquots were run in one ELISA plate, and the recovery percentage was calculated based on the respective uncycled sample. The assay stability assesses whether sample measurements are stable after incubation at room temperature for 24 hours. Three assay kits were included and the same eight samples that were used for the Assay Precision and Reproducibility tests. The acceptance criteria for both tests were 100±20%.

### In Vitro Neutrophil Model

An in vitro neutrophil model was optimized to assess the C3-HNE assay. Primary neutrophils (100.000 cells/well in 96 well-plate) were seeded in type III coated (78 μg/cm2) wells. Activation media (IMDM with LPS 25 or 100 ng/ml) was added to the wells and the cells were incubated for 6 hours. Inactivated neutrophils were included as control. The controls and conditioned media of inactivated and activated neutrophils seeded in type III collagen-coated wells were measured with the C3-HNE assay.

### Clinical Evaluation

### Cohort I

Cohort I originates from the Department of Gastroenterology and Hepatology of Clinical Hospital Centre Zagreb in Croatia. Cohort I includes patients with UC (n=40) and CD (n=85) and healthy donors (n=40) which were obtained from BioVIT (Westerbury, NY, USA). Blood and fecal samples were collected on the day of the visit and stored at −70° C. Information regarding the treatment, demographical data, and disease history were acquired through surveys and medical records. Anthropometric measurements were determined upon inclusion. The Mayo Endoscopic Score (MES, (0: inactive disease; 1: mild activity, 2: moderate activity, 3: severe activity)), full Mayo score, and partial Mayo Score (pMayo, (<2: remission; 2-4: mild activity; 5-7: moderate activity; >7: severe activity)) were used for endoscopic disease activity assessment for patients with UC. The Simple Endoscopic Score for Crohn's disease (SES-CD, (0-2: remission; 3-6: mild disease activity; 7-15: moderate disease activity, >16: severe disease activity)) was used to determine the endoscopic disease activity. Crohn's disease location (L1=ileal, L2=colonic, and L3=ileocolonic) and behavior (B1=luminal, and B2=structuring) were classified by the Montreal classification. Endoscopy was performed within three months after blood sampling. Endoscopic scores for disease activity were recorded and prospectively validated based on routine endoscopy and scoring. Both were classified by an experienced gastroenterologist. The study was approved by the Ethical Committee of University Hospital Centre Zagreb.

To compare the C3-HNE levels between groups, Mann-Whitney U-test or Kruskal-Wallis (Dunn's corrected) were conducted were conducted. The discriminative utility of C3-HNE was evaluated by AUC using ROC curves.

### Cohort II

The study population of Cohort II consists of IBD patients (n=149) that were treated with Infliximab (IFX). Blood samples were acquired at the beginning of the study with two follow-ups. This study included the same healthy samples (n=40) as cohort I. The C3-HNE assay was blindly executed. The study was approved by the Danish Data Protection Agency and The Regional Ethics Committee of Region Hovedstaden, Denmark. Responders were defined as having a score of 0 according to the Physicians Global Assessment determined at Visit 3/3. Additional patient demographical data is pending. To compare the C3-HNE levels between groups, Mann-Whitney U-test was conducted. The discriminative utility of C3-HNE was evaluated by AUC using ROC curves.

### Cohort III

Cohort III was obtained from Herlev/Bispebjerg Hospital, Denmark, including patients with Crohn's Disease (CD) (n=49) and healthy donors (HD) (n=44). Endoscopic and clinical disease activity was assessed using the Ulcerative Colitis Endoscopic Index of Severity (UCEIS) and the full Mayo score. C3-HNE was measured in serum from the 33 patients with active Ulcerative colitis (UC) that were followed from baseline to week 24 and grouped according to response to treatment (defined as a change in total Mayo score from severe/moderate disease at baseline to mild disease/remission at week 24). To compare the C3-HNE levels between groups, Mann-Whitney U-test was conducted. The discriminative utility of C3-HNE was evaluated by AUC using ROC curves.

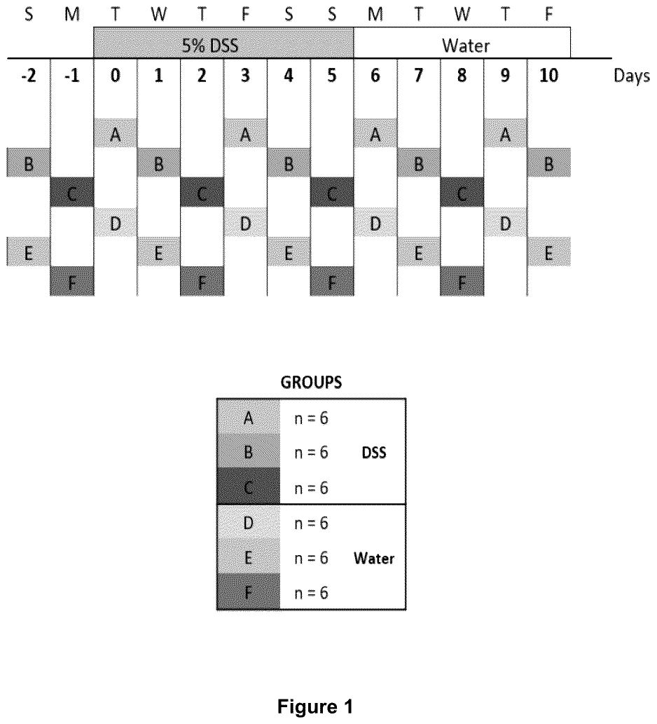

DSS-Induced Colitis Model 12-week male Sprague Dawley (SD) rats (n=36) were divided into three groups (n=6) and treated with 5% (wt/vol) of DSS (Sigma-Aldrich, Denmark) in drinking water for six days to induce colitis (Groups A, B and C). Then DSS was omitted, and rats received normal water for five days (Wirtz et al., 2007). Matched control rats were also divided into three groups (n=6) and received only regular drinking water (Groups: D, E and F). The disease activity index (DAI) score was monitored daily, and blood was drawn according to the six groups (n=6). Termination was performed for histology of the tissues on day eight for D and F groups, day nine for A and D groups, and day ten for B and E groups as seen in FIG. 1.

### Cleavage of Type III Collagen

The purpose of the experimental cleavage was to assess whether HNE can release the desired fragment from a fibroblast-deposited ECM. Due to the tightly packed structure of collagen helices, the collagenases MMP-8 and -13 were used for pre-cleavage to unwind the tightly packed triple-helix. The experiment was performed on ice and the buffers used were:
1. HNE assay buffer (100 mM Tris-HCl, 500 mM NaCl, pH 7.5) for Active HNE (Abcam)
2. MMP assay buffer (50 mM Tris-HCl, 200 mM NaCl, 10 mM CaCl₂), 100 μM ZnCl, pH 7.5) for MMPs.

Scar-in-a-Jar (SIAJ) is a model that stimulates fibrosis in vitro, where fibroblast cells (derived from the GI tract) deposit ECM into plastic wells.

Human primary intestinal fibroblasts purchased from Cell biologics (Chicago, Illinois, USA) (cat. no. H-6025) were grown to confluence and seeded in passage 6-8 at a density of 30,000 cells/well in 48-well plates in a high serum medium (10% fetal bovine serum (FBS) (cat. no. F7524, Sigma-Aldrich, St. Louise, Missouri, USA) in Dulbecco's modified eagle medium (DMEM)+Glutamax (cat. no. 31966, Gibco, Life Technologies, Carlsbad, California, USA)) on day-2. On day-1, the cells were serum-starved in a low serum medium (0.4% FBS DMEM), avoiding interference with biomarker measurements. Upon induction of fibrogenesis on day 0, cells were cultured in a low serum medium containing ficoll-70 (112.5 mg/mL, cat. no. F2878, Sigma-Aldrich, St. Louise, Missouri, USA) and −400 (75 mg/mL, cat. no. F4375, Sigma-Aldrich, St. Louise, Missouri, USA), supplemented with 1.0% L-ascorbic acid, phosphate magnesium salt, n-hydrate (cat. no. 013-19,641, Wako, Osaka, Japan) without or with stimulation of 20 ng/ml TGF-β1 (cat no. 100-B-010/CF, R&D system, Minneapolis, Minnesota, USA). Cell culturing occurred by incubation at 37° C. with 95% O₂ and 5% CO₂ for 12 days, exchanging for a freshly prepared medium at days 0, 4, and 8.

The wells were decellularized and incubated with HNE, or HNE and Matrix metalloproteinases—MMP8 (RnD systems, 904-MP, lot CLF0721041) and MMP13 (RnD systems, 511-MM, lot CJT1221111), or just MMP8 and MMP13. The HNE reactions were incubated for 72 hours at 37° C. and stopped with the addition of 2 μl of AEBSF (Thermo Fischer) inhibitor at a final concentration of 1 mM. The Matrix metalloproteinases (MMPs) were activated with 4-aminophenylmercuric acetate (APMA) in MMP assay buffer for 2 hours at 37° C. (1 mM for 100 μg/ml protease) before the incubation with collagen type III. Then, MMPs reactions were incubated for 24 hours at 37° C. and stopped with EDTA at final concentration of 1 mM. A control of uncleaved type III collagen was incubated for 72 hours at 37° C. The protease-to-protein ratio was 1:100, and the concentration for each reagent can be seen in Table 1.

TABLE 1
Working stock concentration for the HNE and MMP reactions
to cleave type III collagen from matrix of SIAJ model.

| | Working | Final |
|---|---|---|
| | stock | concentration |
| | concen- | in reaction |
| | tration | volume |
| (1) Matrix + HNE | | |
| HNE | 100 μg/ml | 1 μg/ml (0.4 μg) |
| (2) Matrix + MMP8 + MMP13 | | |
| MMP-8 | 40 μg/ml | 1 μg/ml (0.4 μg) |
| MMP-13 | 40 μg/ml | 1 μg/ml (0.4 μg) |
| (3) Matrix + MMP8 + MMP13 + HNE | | |
| HNE | 100 μg/ml | 1 μg/ml (0.4 μg) |
| MMP-8 | 40 μg/ml | 1 μg/ml (0.4 μg) |
| MMP-13 | 40 μg/ml | 1 μg/ml (0.4 μg) |
| (4) Buffer ctrl | | |
| HNE | 100 μg/ml | 1 μg/ml (0.4 μg) |
| MMP-8 | 40 μg/ml | 1 μg/ml (0.4 μg) |
| MMP-13 | 40 μg/ml | 1 μg/ml (0.4 μg) |

### Results

This section describes the results obtained throughout the assay development and the assessment of technical performance for the C3-HNE biomarker assay. Subsequently, preliminary evaluation and biological relevance was investigated in serum from patients with IBD (UC and CD) in one pre-clinical and three clinical studies. Furthermore, as a proof of concept, fibroblast-deposited type III collagen was cleaved.

### The C3-HNE Antibody is Highly Specific for the Intended Fragment

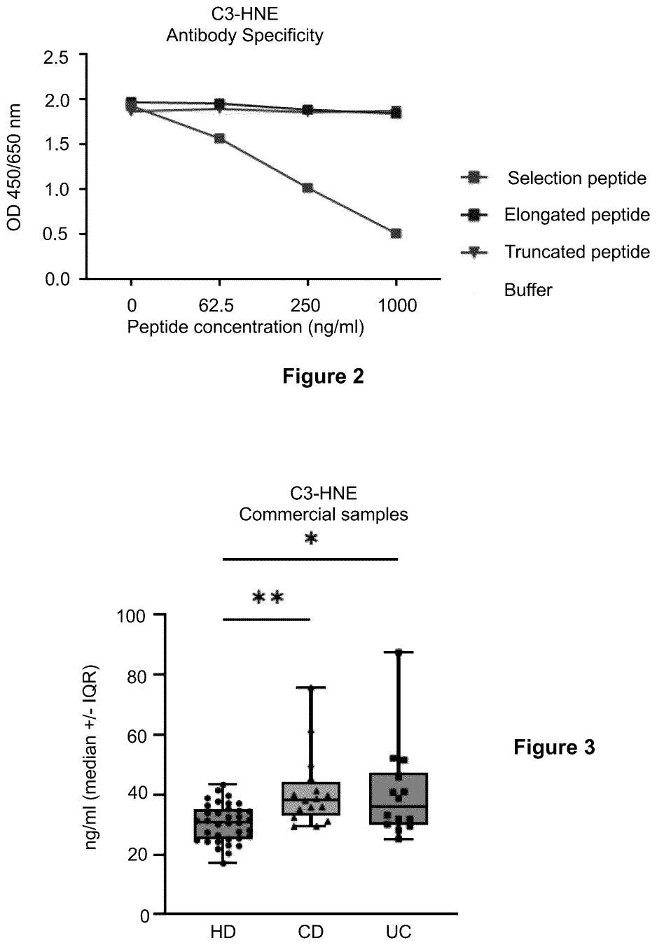

The specificity of the neo-epitope C3-HNE was evaluated by the reactivity of the mAb against the selection (standard) peptide (PGKNGERGGP (SEQ ID NO: 1)), the elongated peptide (APGKNGERGGP (SEQ ID NO: 2)), and the truncated peptide (GKNGERGGP (SEQ ID NO: 3)). The selection peptide representing the neo-epitope inhibited the signal from the reaction between the coater peptide and the antibody, dose-dependently. Upon incubation of the mAb with the elongated and truncated peptides, no reactivity was exhibited. Thus, the selection peptide solely competed for the antibody binding. These results show that the mAb is highly specific to the neo-epitope of interest (FIG. 2).

### C3-HNE is Elevated in IBD Patients Compared to Healthy Donors

Biological relevance was assessed in commercial IBD samples and healthy donors before the technical validation of the assay. Samples were incubated for 1 hour, with a coater concentration of 0.5 ng/ml and a mAb concentration of 15 ng/ml. C3-HNE was elevated significantly in both samples with CD (n=16) and UC (n=14) compared to healthy (n=32) samples (38.2 ng/ml [32.94, 44.30] vs 35.86 ng/ml [29.80, 47.16] vs. 30.85 ng/ml [25.10, 34.93]) (p=0.002 and p=0.036) (FIG. 3).

### C3-HNE is a Technically Robust ELISA Assay

### Measurement Limits

The LLOB is the highest quantile value in a blank sample. For the C3-HNE assay the blank sample is the assay buffer: 50 mM PBS-BTB, 8 g NaCl, pH 7.4, also known as background. The LLOB was determined by performing a single run of 60 replicates of the assay buffer. It was calculated as the average mean of the measurements: 3.72 ng/ml. Additionally, the LLOQ is the lowest limit at which the assay can provide quantitative measurements. The LLOQ was conducted with four human serum samples of low concentration (double determination and three duplicates) in five independent runs. The LLOQ was established to be 7.00 ng/ml.

### Assay Linearity

The minimum required dilution (MRD) and dilution recovery were determined by the linearity that was validated during two-fold dilution of human serum samples. For the dilution recovery, four human serum samples of high concentration were diluted in the assay buffer and the linearity was repeated three times, once per day. The acceptance criteria for the dilution recovery were 100±20% and the samples showed an acceptable dilution recovery for 4-fold dilution. The 8-fold dilution recovery was not within the acceptance criteria of at least 75% of the samples. When serial diluting, the concentrations were decreased from the dilution 1+0 to 1+2, indicating no matrix effects. Thus, the MRD was determined to be 1+0 (undiluted samples).

### Assay Accuracy

The accuracy of the assay was determined by the matrix-matrix spiking recovery. Three samples with low concentration (around IC20) were spiked in three samples with high concentrations (above IC50). Each sample set had a matrix-matrix spike-in of the ratios 100:0, 75:25, 50:50, 25:75, and 0:100 of high to low concentration samples, respectively. The accepted recovery criteria were 100±20% for at least 75% of the samples.

### Assay Precision and Reproducibility

Inter-intra assay determined the robustness of the assay and the precision of the measured concentrations within the same plate or between plates, in 10 independent runs. The samples used were eight serum samples and two QCs covering the assay's measurement range. The robustness of the assay was calculated as the coefficient of variation in percentage (CV %) of the sample measurements. The 11-point standard curve was robust and had a recovery rate of 100% for the first seven points, whereas the eighth point of 7.81 ng/ml failed (60% recovery). However, the curve fit was best with 11-points and remained as such. The recovery rate for all the parameters was below 10% and specifically the average value of IC50 was ~58 and the average value of slope was ~1.07. The inter assay variation had a range between 3-12% and the intra assay variation had a range of 4-9%, which were within the acceptance criteria.

### Assay Stability

The assay stability was tested by a freeze-thaw test and a kit stability test. In the freeze-thaw test, the analyte was exposed to five freeze-thaw cycles. The recovery percentage was within the accepted range of 100±20%. The kit stability was tested in three assay runs for the same eight samples that were used for the Assay Precision and Reproducibility tests. The assay reagents of 3 kits were left for 24 hours at room temperature. The results showed that the recovery percentage was within the accepted range of 100±20% for all samples. Overall, the assay exhibited good stability.

### Assay Interference

The assay interference was tested by spiking haemoglobin, biotin, and lipids, in different concentrations into three serum samples. The analysis showed no interference between all substances and the serum samples. The recovery percentages were all accepted within the range of 100±20%.

### Final Assay Parameters

The results of the technical validation show that the C3-HNE assay is technically robust and can be used for testing in human serum samples. A summary of all technical validation parameters is presented in Table 2. These characteristics should be considered when measuring human serum samples with the C3-HNE assay.

TABLE 2
Technical validation parameters of the assay C3-HNE.

| ASSAY PARAMETERS | RESULT | |
|---|---|---|
| ELISA format | Competitive | |
| | 50 mM PBS-BTB, 8 g NaCl, pH | |
| Incubation buffer | 7.4 | |
| Incubation time | 20 hours at 4° C. | |
| Intended matrix (MRD) | Serum (undiluted) | |
| Measurement range (LLOQ-ULOQ) | 15.62-965.59 | ng/ml |
| IC50 | 58.11 | ng/ml |
| Slope | 1.08 | |
| LLOB | 3.72 | ng/ml |

| | |
|---|---|
| Intra-assay variation (CV %) | 4-9% |
| Inter-assay variation (CV %) | 3-12% |
| Dilution recovery in serum (1 + 3) | 91-108% |
| Freeze-thaw stability (5 cycles) | 87-107% |
| Kit Stability 24 hours at 20° C. | 99-119% |
| Interference hemoglobin, low/high^{a} | 94-96%/87-98% |
| Interference lipid, low/high^{b} | 96-105%/94-104% |
| Interference biotin^{c} | 84-107% |

^{a}2.5/5 mg/ml,
^{b}1.5/5 mg/ml,
^{c}5/100 ng/ml

C3-HNE is Elevated in Patients with IBD Compared to Healthy Donors

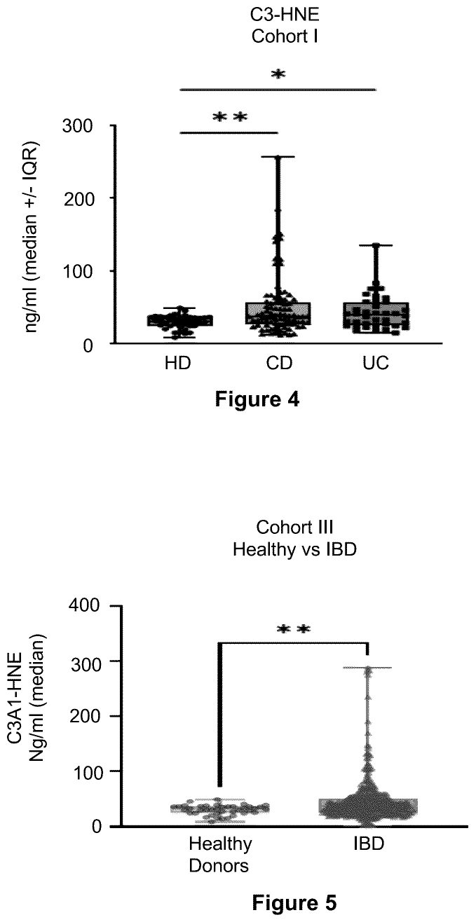

The biological utility of C3-HNE was evaluated in serum samples from cohort I, which included patients with CD (n=40), UC (n=85) and healthy controls (n=40). C3-HNE levels were significantly higher in patients with CD and UC compared to healthy controls (37.44 ng/ml [27.07, 57.44] vs 39.8 ng/ml [25.45, 57.25] vs 31.2 ng/ml [24.48, 36.54]) (p=0.007 and p=0.043) (FIG. 4). All quantified levels of C3-HNE were above LLOQ. These results suggest that the generation of the C3-HNE fragment is associated with IBD.

The biological relevance of C3-HNE was validated in serum samples from cohort II, including patients with IBD (n=149) that were treated with infliximab. This study included the same healthy samples (n=40) as cohort I. C3-HNE levels were significantly higher in patients with IBD compared to healthy controls (36.64 ng/ml [25.17, 50.71] vs 31.2 ng/ml [24.48, 36.54]) (p=0.003) (FIG. 5). All quantified levels of C3-HNE were above LLOQ. These results suggest that the generation of the C3-HNE fragment is associated with IBD and demonstrates biomarker potential.

### C3-HNE is a Potential Marker for Early Stages of Clinically Apparent Mucosal Damage

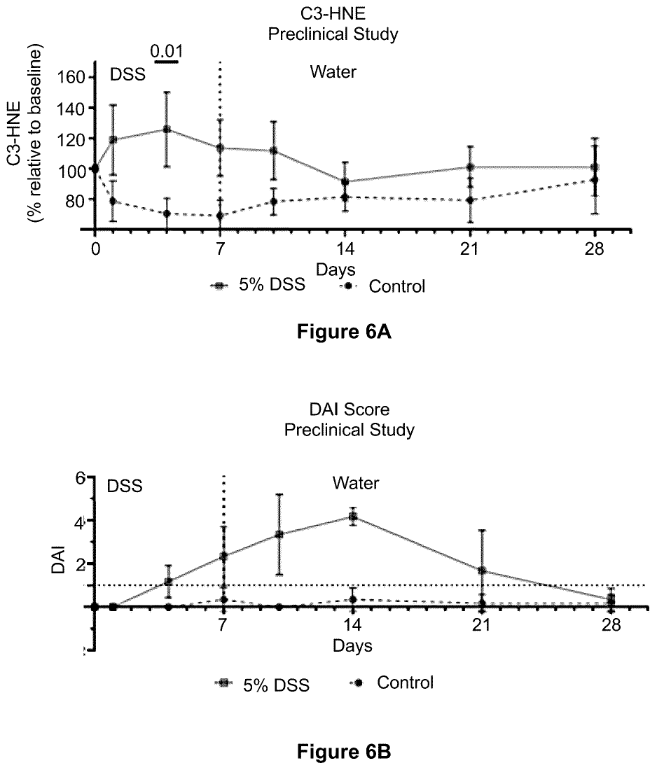

The C3-HNE levels were significantly increased during the DSS treatment, whereas the control group demonstrated decreased levels of the biomarker. During the induction of colitis, C3-HNE levels increase earlier (day 1) in the timeline than the elevation of the disease activity index (DAI) (day 7). Upon omission of DSS, the DAI score slowly increases until day 14, where it reaches the maximum, whereas the levels of C3-HNE drop rapidly to the same level as the control group on day 14 (FIG. 6). Thus, the results suggest that the C3-HNE biomarker reflects early stages of clinically apparent mucosal damage during experimental colitis.

The C3-HNE Fragment is Released Upon HNE Cleavage from Fibroblast-Deposited ECM

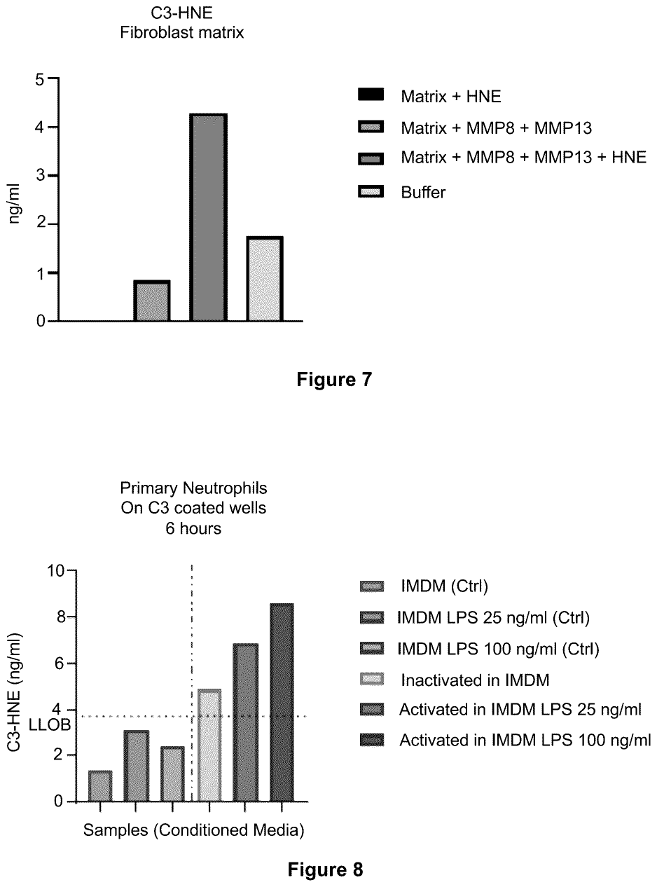

The measured C3-HNE was elevated in scar in a jar (SIAJ) matrix cleaved with MMP-8, MMP-13 and HNE when compared to the negative controls (Matrix+HNE and Matrix+MMP-8+MMP-13) (FIG. 7). All measurements were below LLOQ (established for human serum).

### Discriminative Ability of C3-HNE in Commercial Samples, Cohort I and Cohort II.

Based on the ROC analysis, the biomarker can significantly discriminate between healthy donors (HD) and Crohn's disease (CD) or ulcerative colitis (UC) (P<0.001 and p=0.01) in commercial samples, between HD and CD or UC (P<0.01 and p=0.01) in Cohort I and between HD and IBD (P<0.05) in Cohort II (Table 3).

TABLE 3
ROC curve analysis results depicting the discriminative
ability of C3-HNE between healthy and diseased donors.

| | | Sensitivity | | p- |
|---|---|---|---|---|
| C3-HNE | AUC [95% CV] | (%) | Specificity (%) | value |
| Commercial | | | | |
| Samples | | | | |
| HD vs. CD | 0.80 [0.67-0.92] | 56.25 | 88.89 | <0.001 |
| HD vs. UC | 0.72 [0.56-0.88] | 50.00 | 88.89 | =0.01 |
| Cohort I | | | | |
| HD vs. CD | 0.66 [0.57-0.75] | 47.06 | 92.50 | <0.01 |
| HD vs. UC | 0.66 [0.52-0.79] | 50.00 | 95.00 | =0.01 |
| Cohort II | | | | |
| HD vs IBD | 0.61 [0.52-0.69] | 43.95 | 92.50 | <0.05 |

C3-HNE fragment is released upon LPS activation from Primary Neutrophils seeded on type III collagen.

The measured C3-HNE levels were elevated in primary neutrophils that were activated with LPS for 6 hours. Although insignificant, an interesting trend was noted for neutrophils activated with LPS, where the C3-HNE biomarker levels increased from the stimulation of ng/ml LPS compared to 100 ng/ml LPS on the type III collagen coated wells. (FIG. 8)

C3-HNE is a Potential Candidate for the Monitoring of Treatment Response in Patients with IBD.

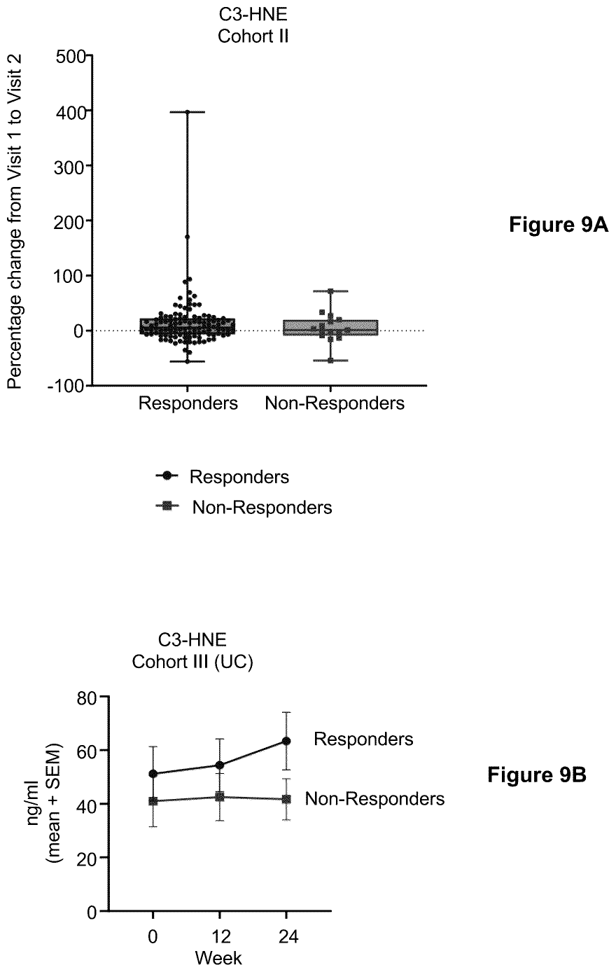

The percentile range for C3-HNE from visit 1 to visit 3 is higher for the responders compared to non-responders, although not significant difference was observed (FIG. 9.A). For cohort III, responders to treatment at week 24 presented elevated levels of C3-HNE compared with non-responders, while no significant differences were observed between the groups. (FIG. 9.B, Table 4).

In cohort II, C3-HNE levels were elevated in non-responders compared to responders (ng/ml [IQR]: 54.47 [2642, 70.2] vs 43.8 [23.2, 60.8], p=0.1) (Table 4). However, no significant difference was observed between the patients grouped according to treatment.

In Cohort III, when patients were stratified according to response to treatment, C3-HNE was elevated in responders compared to non-responders (ng/ml [IQR]: 47.5 [30.8, 70.9] vs 31.4 [23.2, 58.2], p=0.15), although no significant difference was observed (Table 4).

TABLE 4
C3-HNE levels of Cohort II (grouped by response to treatment), and Cohort
II (grouped in HD and responders & non-responders IBD patients) and
ROC curve analysis results depicting the discriminative ability of
C3-HNE between healthy and diseased (IBD) donors for Cohort II.

| Visit | Patients | AUC | Sensitivity (%) | Specificity (%) | Cut-off | p-value |
|---|---|---|---|---|---|---|
| 1 | Responders vs. | 0.527 | 32.60 | 82.60 | 26.538 | 0.670 |
| | Non-Responders | | | | | |
| 2 | Responders vs. | 0.541 | 23.66 | 85.71 | 24.360 | 0.586 |
| | Non-Responders | | | | | |
| 3 | Responders vs. | 0.510 | 20.00 | 85.71 | 23.514 | 0.891 |
| | Non-Responders | | | | | |

Cohort II

| Visit | Responders | Non-Responders | p-value |
|---|---|---|---|
| 1-3 | 43.8 [23.2, 60.8] | 54.47 [2642, 70.2] | 0.100 |

Cohort III

| Week | Score | Responders | Non-Responders | p-value |
|---|---|---|---|---|
| 0-24 | tMayo | 47.5 [30.8, 70.9] | 31.4 [23.2, 58.2] | 0.155 |

Data is presented as medians with IQR.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used to mean ‘including or consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

Bradbury, A. R. M, Trinklein, N. D., Thie, H., Wilkinson, I. C., Tandon A. K, Anderson, S, . . . & Dübelp, S. (2018) When monoclonal antibodies are not monospecific: Hybridomas frequently express additional functional variable regions. MAbs. 2018 May-June; (4): 539-546. doi: 10.1080/19420862.2018.1445456
Curciarello, R., Sobande, T., Jones, S., Giuffrida, P., Di Sabatino, A., Docena, G. H., . . . & Kok, K. (2020). Human neutrophil elastase proteolytic activity in ulcerative colitis favors the loss of function of therapeutic monoclonal antibodies. Journal of Inflammation Research, 13, 233.
Fischbach, W., Becker, W., Mössner, J., Ohlemüller, H., Koch, W., & Börner, W. (1987). Leucocyte elastase in chronic inflammatory bowel diseases: a marker of inflammatory activity?. Digestion, 37 (2), 88-95.
Fu, Z., Thorpe, M., Akula, S., Chahal, G., & Hellman, L. T. (2018). Extended cleavage specificity of human neutrophil elastase, human proteinase 3, and their distant ortholog clawed frog PR3—three elastases with similar primary but different extended specificities and stability. Frontiers in Immunology, 2387.
Gefter, M. L., Margulies, D. H., & Scharff, M. D. (1977). A simple method for polyethylene glycol-promoted hybridization of mouse myeloma cells. Somatic cell genetics, 3(2), 231-236.
Graham, D. B., & Xavier, R. J. (2020). Pathway paradigms revealed from the genetics of inflammatory bowel disease. Nature, 578 (7796), 527-539.
Graham, M. F., Diegelmann, R. F., Elson, C. O., Lindblad, W. J., Gotschalk, N., Gay, S., & Gay, R. (1988). Collagen content and types in the intestinal strictures of Crohn's disease. Gastroenterology, 94 (2), 257-265.
Hansberry, D. R., Shah, K., Agarwal, P., & Agarwal, N. (2017). Fecal myeloperoxidase as a biomarker for inflammatory bowel disease. Cureus, 9 (1).
Jostins, L., Ripke, S., Weersma, R. K., Duerr, R. H., McGovern, D. P., Hui, K. Y., . . . & Cho, J. H. (2012). Host-microbe interactions have shaped the genetic architecture of inflammatory bowel disease. Nature, 491 (7422), 119-124.
Kehlet, S. N., Willumsen, N., Armbrecht, G., Dietzel, R., Brix, S., Henriksen, K., & Karsdal, M. A. (2018). Age-related collagen turnover of the interstitial matrix and basement membrane: Implications of age- and sex-dependent remodeling of the extracellular matrix. PLOS One, 13(3), e0194458.
Kong, S., Zhang, Y. H., & Zhang, W. (2018). Regulation of intestinal epithelial cells properties and functions by amino acids. BioMed research international, 2018.
Kriaa, A., Jablaoui, A., Rhimi, S., Soussou, S., Mkaouar, H., Mariaule, V., . . . & Rhimi, M. (2021). SP-1, a Serine Protease from the Gut Microbiota, Influences Colitis and Drives Intestinal Dysbiosis in Mice. Cells, 10 (10), 2658.
Maillet, N. (2020). Rapid Peptides Generator: fast and efficient in silico protein digestion. NAR Genomics and Bioinformatics, 2 (1), Iqz004.
Menegazzi, R., Decleva, E., & Dri, P. (2012). Killing by neutrophil extracellular traps: fact or folklore?. Blood, The Journal of the American Society of Hematology, 119 (5), 1214-1216.
Mimura, Y., Church, S., Ghirlando, R., Ashton, P. R., Dong, S., Goodall, M., . . . & Jefferis, R. (2000). The influence of glycosylation on the thermal stability and effector function expression of human IgG1-Fc: properties of a series of truncated glycoforms. Molecular immunology, 37 (12-13), 697-706.
Pahwa, R., Goyal, A., & Jialal, I. (2021). Chronic inflammation. StatPearls [Internet].
Qiu, P., Ishimoto, T., Fu, L., Zhang, J., Zhang, Z., & Liu, Y. (2022). The gut microbiota in inflammatory bowel disease. Frontiers in Cellular and Infection Microbiology, 102.
Saez, A., Herrero-Fernandez, B., Gomez-Bris, R., Sánchez-Martinez, H., & Gonzalez-Granado, J. M. (2023). Pathophysiology of Inflammatory Bowel Disease: Innate Immune System. International Journal of Molecular Sciences, 24 (2), 1526.
Schmid, M., Fellermann, K., Fritz, P., Wiedow, O., Stange, E. F., & Wehkamp, J. (2007). Attenuated induction of epithelial and leukocyte serine antiproteases elafin and secretory leukocyte protease inhibitor in Crohn's disease. Journal of leukocyte biology, 81 (4), 907-915.
Signore, A. (2013). About inflammation and infection. EJNMMI research, 3(1), 8.
Wirtz, S., Neufert, C., Weigmann, B., & Neurath, M. F. (2007). Chemically induced mouse models of intestinal inflammation. Nature protocols, 2 (3), 541-546.
Yuen, J., Pluthero, F. G., Douda, D. N., Riedl, M., Cherry, A., Ulanova, M., . . . & Licht, C. (2016). NETosing neutrophils activate complement both on their own NETs and bacteria via alternative and non-alternative pathways. Frontiers in immunology, 7, 137.

1: A method of immunoassay, said method comprising;
i) contacting a patient sample with a monoclonal antibody that specifically binds to the N-terminus amino acid sequence PGKNGERGGP (SEQ ID NO: 1); and
ii) detecting and determining the amount of binding between said monoclonal antibody and peptides in the sample.

2: The method as claimed in claim 1, wherein the method is a method of immunoassay for detecting and/or monitoring inflammatory bowel disease or a particular level of severity thereof in a patient, the method further comprising;
ii) correlating said amount of binding with values associated with normal healthy subjects and/or values associated with known disease severity and/or values obtained from said patient at a previous time point and/or with a predetermined cut-off value.

3: The method as claimed in claim 2, wherein the inflammatory bowel disease is Crohn's disease or ulcerative colitis.

4: The method as claimed in claim 1, wherein the patient sample is selected from blood, plasma or serum.

5: The method as claimed in claim 1, wherein the monoclonal antibody specifically binds to the N-terminus amino acid sequence PGKNGERGGP (SEQ ID NO: 1) and does not specifically bind to a peptide having the N-terminus amino acid sequence APGKNGERGGP(SEQ ID NO: 2).

6: The method as claimed in claim 1, wherein the monoclonal antibody specifically binds to the N-terminus amino acid sequence PGKNGERGGP (SEQ ID No: 1) and does not specifically bind to a peptide having the N-terminus amino acid sequence GKNGERGGP (SEQ ID NO: 3).

7: The method as claimed in claim 1, wherein the monoclonal antibody is raised against a synthetic peptide having the N-terminus amino acid sequence PGKNGERGGP (SEQ ID NO: 1).

8: The method as claimed in claim 1, wherein the immunoassay is a competition assay or a sandwich assay.

9: The method as claimed in claim 1, wherein the immunoassay is a radio-immunoassay or an enzyme-linked immunosorbent assay.

10: A monoclonal antibody that specifically recognises the N-terminus amino acid sequence PGKNGERGGP(SEQ ID NO: 1).

11: The monoclonal antibody as claimed in claim 10 [13], wherein the monoclonal antibody specifically binds to the N-terminus amino acid sequence PGKNGERGGP (SEQ ID NO: 1) and does not specifically bind to a peptide having the N-terminus amino acid sequence APGKNGERGGP (SEQ ID NO: 2).

12: The monoclonal antibody as claimed in claim 10, wherein the monoclonal antibody specifically binds to the N-terminus amino acid sequence PGKNGERGGP(SEQ ID NO: 1) and does not specifically bind to a peptide having the N-terminus amino acid sequence GKNGERGGP (SEQ ID NO: 3).

13: The monoclonal antibody as claimed in 10, wherein the monoclonal antibody is raised against a synthetic peptide having the N-terminus amino acid sequence PGKNGERGGP (SEQ ID NO: 1).

14: An immunoassay kit comprising the monoclonal antibody as claimed in claim 10, and at least one of:
a streptavidin coated well plate;
a biotinylated peptide PGKNGERGGP-L-Biotin (SEQ ID NO: 20), wherein L is an optional linker;
a secondary antibody for use in a sandwich immunoassay;
a calibrator protein comprising the N-terminus amino acid sequence PGKNGERGGP (SEQ ID NO: 1);
an antibody biotinylation kit;
an antibody HRP labelling kit; or
an antibody radiolabelling kit.
